(19) United States
(12) Patent Application Publication
(10) Pub. No.: US 2005/0228238 A1
(43) Pub. Date: Oct. 13, 2005

(54) Patient parameter automatic acquisition system

(76) Inventor: Arnold Monitzer, Wayne, PA (US)

Correspondence Address: Alexander J. Burke;Intellectual Property Department, 5th Floor, 170 Wood Avenue South, Iselin, NJ, 08830, US

(21) Appl. No.: 10/872,677

(22) Filed: Jun. 21, 2004

Related U.S. Application Data
(60) Provisional application No. 60/561,186, filed on Apr. 9, 2004.

(51) Int. Cl. A61B 5/00; A61B 5/04; A61B 5/02

(52) U.S. Cl. 600/300; 128/903; 600/549; 600/544; 600/509; 600/485

(57) ABSTRACT

A system automatically acquires and associates patient parameter ancillary information with patient identity data to reduce manual data entry and resulting medical error. A patient parameter processing system includes a transceiver for automatically wirelessly acquiring, from a patient attached tag, data enabling derivation of information identifying the patient. An acquisition processor automatically acquires a patient parameter from a device attached to a patient. A data processor automatically transfers both the acquired patient parameter and derived patient identification information in conjunction, to a system for generating patient related records to ensure the acquired patient parameter is associated with a correct patient.

This is a non-provisional application of provisional application Ser. No. 60/561,186 by A. Monitzer filed Apr. 9, 2004.

### FIELD OF THE INVENTION

This invention concerns a system for automating patient parameter acquisition and patient identification.

### BACKGROUND INFORMATION

Existing patient parameter acquisition systems acquire and process parameters such as weight measurements, blood pressure, blood oxygen saturation, respiration, ECGs, pulse rate, temperature and other parameters Existing patient parameter acquisition systems exhibit a number of problems that occur because of the need for a user to perform manual data processing tasks associated with patient parameter acquisition. Such manual data processing tasks include entering ancillary data identifying a patient, patient financial information or demographic information as well as other types of data such as test conditions (including patient posture, movement etc.), data identifying parameters being acquired, a patient medical condition during parameter acquisition, medications administered and other contextual information affecting parameter values acquired or their interpretation, for example. The manual entry of ancillary data typically occurs via a user interface of a patient parameter data acquisition device.

User manual entry of patient parameter ancillary data is burdensome and increases the risk of error (perhaps affecting a physician order for administration of medication to a patient, for example) resulting from user entry of incorrect data. The need for manual data entry also delays processing of patient parameters which reduces the number of patients a clinician is able to handle in a given time period and reduces throughput and patient care efficiency. These problems and associated problems are addressed by a system according to invention principles.

### SUMMARY OF THE INVENTION

A system automatically acquires patient parameter ancillary information (such as patient identification information, healthcare insurance information or demographic information, for example) and an associated patient parameter (such as patient weight information, for example) for communication to a computerized physician medication order entry system or electronic patient record. A patient parameter processing system includes a transceiver for automatically wirelessly acquiring, from a patient attached tag, data enabling derivation of information identifying the patient. An acquisition processor automatically acquires a patient parameter from a device attached to a patient. A data processor automatically transfers both the acquired patient parameter and derived patient identification information in conjunction, to a system for generating patient related records to ensure the acquired patient parameter is associated with a correct patient.

### BRIEF DESCRIPTION OF THE DRAWING

FIG. 1 shows a patient parameter processing system, according to invention principles.

FIG. 2 shows a flowchart of steps for processing patient parameters employed by the system of FIG. 1, according to invention principles.

FIG. 3 shows a flowchart of steps involved in acquiring and processing patient parameters, according to invention principles.

### DETAILED DESCRIPTION OF INVENTION

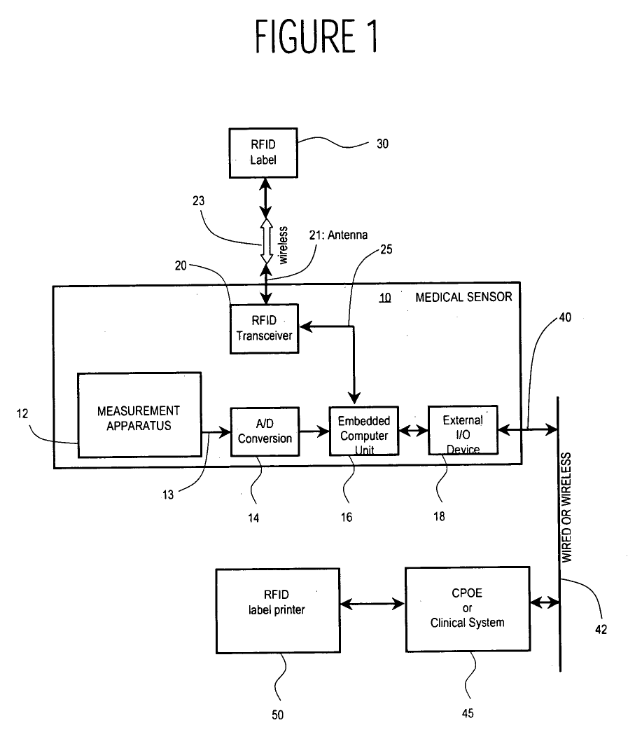

FIG. 1 shows a system that automatically acquires and processes patient parameters and associated ancillary data. Patient parameters processed include weight measurements, blood pressure, blood oxygen saturation, respiration, ECGs, pulse rate, temperature and other parameters Associated ancillary data includes, data identifying a patient, patient financial information or demographic information but may also include other types of data such as test conditions (including patient posture, movement etc.), data identifying parameters being acquired, a patient medical condition during parameter acquisition, medications administered and other contextual information affecting parameter values acquired or their interpretation, for example.

A computerized physician order entry (CPOE) system is used to help physicians reduce risk of medical errors resulting from manual steps in acquiring data used in ordering treatment for a patient. A medication dose for a patient in a computerized order entry system is typically determined based on a patient weight. Patient weight needs to be measured exactly and entered accurately in an order entry system in order to reduce risk of error in prescribing a medication dose. However such data entry steps in known systems are manual and vulnerable to human error.

Although the preferred embodiment is described in connection with a medical scale (patient weighing device) and a patient weight measurement parameter, this is exemplary only. Other types of patient parameter acquisition device and other types of parameter and associated ancillary data may be processed by a system according to invention principles. In the FIG. 1 system a patient parameter acquisition device (an advantageously adapted medical weighing scale) acquires and digitizes patient weight information and transfers the information to a computerized physician order entry (CPOE) system. The scale may be embodied within a patient bed, a patient examination table, a patient chair and a couch used to support said patient, for example. In addition, a patient identifier is automatically acquired. This enables a patient to be automatically identified and authenticated by the automatic weighing scale. The patient identifier is acquired from a printed passive radio frequency identification device (RFID) Tag that the patient received at check-in. (RFID is described at www.rfidjournal.com and associated web pages.) For this purpose the system employs a RFID reader to identify a patient that is currently located on the scale.

The system advantageously automates an error prone manual step of entering patient identification information and associating the identification information with a corresponding patient parameter for use in ensuring and validating that accurate treatment orders are placed for a patient in a physician order entry process, for example. The system eliminates manual steps involved in entering a patient parameter, e.g., a patient weight measurement value, as well as in entering patient identification data (and other ancillary data) and in linking the entered information to a patient record. Thereby, the risk of making errors in prescribing patient medication is advantageously reduced. The FIG. 1 system employs an advantageously modified medical weighing scale, a physician order entry or Clinical system that electronically manages order information together with RFID tags and an RFID tag printer. An RFID tag is created to provide patient identification information for a particular patient that corresponds to a physical or electronic patient record folder associated with the patient through the care process.

Existing medical weighing scales typically fail to provide an external interface to a computerized physician order entry system and thereby require manual data entry of a weight measurement value into the order entry system. Existing weighing scales also do not support automatic detection of ancillary data associated with a patient weight measurement value, e.g. patient identity or linkage between identity and the measurement value. Existing weighing scales also do not support the automatic acquisition of patient identification information and association of the identification information with a corresponding patient parameter (weight value).

A processor as used herein is a device and/or set of machine-readable instructions for performing tasks. As used herein, a processor comprises any one or combination of, hardware, firmware, and/or software. A processor acts upon information by manipulating, analyzing, modifying, converting or transmitting information for use by an executable procedure or an information device, and/or by routing the information to an output device. A processor may use or comprise the capabilities of a controller or microprocessor, for example. A display generator is a known element comprising electronic circuitry or software or a combination of both for generating display images or portions thereof. An executable application comprises code or machine readable instruction for implementing predetermined functions including those of an operating system, healthcare information system or other information processing system, for example, in response user command or input.

In the FIG. 1 system, patient parameter acquisition device 10 is a medical weighing scale (or an acquisition device for acquiring other types of patient parameter in another embodiment). Parameter acquisition device 10 includes measurement apparatus 12, analog/digital A/D conversion unit 14, embedded computer unit 16, external interface I/O device 18 and RFID transceiver 20. Parameter acquisition device 10 advantageously converts an analog measurement 13 into digital computer readable form and identifies an object tested via data received from RFID transceiver 20. In this exemplary description, measurement apparatus 12 measures the weight of a patient and provides an analog signal 13 representing the measured weight to A/D conversion unit 14.

RFID tags and transceivers such as devices 20 and 30 are known devices used for applications such as tracking goods during transportation for logistics purposes, asset tracking, and just in time production, for example. RFID transceiver 20 comprises an interrogator or reader with an antenna 21. Transceiver 20 transmits electromagnetic waves 23 that form a magnetic field when they couple with an antenna of RFID tag 30. Passive RFID tag 30 draws power from the magnetic field and uses it to power internal circuits to transmit to transceiver 20. RFID transceiver 20 converts received transmitted signals from tag 30 into digital data 25 provided to embedded computer unit 16.

Embedded computer 16 processes a digitized measured weight value received from A/D conversion unit 14 as well as associated ancillary data 25 received from RFID tag 30 via RFID Transceiver 20. Embedded computer 16 advantageously links the measured weight value of the patient received from A/D unit 14 with ancillary data 25 comprising patient identification information from RFID tag 30. In other embodiments ancillary data from tag 30 may comprise patient financial information or demographic information but may also include other types of data such as test conditions (including patient posture, movement etc.), data identifying parameters being acquired, a patient medical condition during parameter acquisition, medications administered and other contextual information affecting parameter values acquired or their interpretation, for example. In another embodiment, computerized physician order entry system (or clinical system) 45 creates the link between the patient measured weight value and patient identification information. In such an embodiment, embedded computer 16 serves as an I/O gateway and does not combine the information.

Embedded computer 16 uses external interface I/O device 18 to communicate data 40 representing the patient parameter and ancillary data as well as the link between them (specifically the linked patient measured weight value and patient identification information) to the physician order entry system (or clinical system) 45. External interface I/O device 18 communicates between parameter acquisition device 10 and the physician order entry system (or clinical system) 45 using Internet Protocol (IP) over wired or wireless communication network 42. However, device 18 may also employ other protocols such as Open Systems Interconnect (OSI) standard, e.g. X.25 compatible protocol. The physician order entry system (or clinical system) 45 stores the measured weight value and patient identification information in the appropriate patient record in a database. The physician order entry system 45 is also interfaced with RFID tag printer 50 to create a RFID tag 30 for each patient during hospital admission.

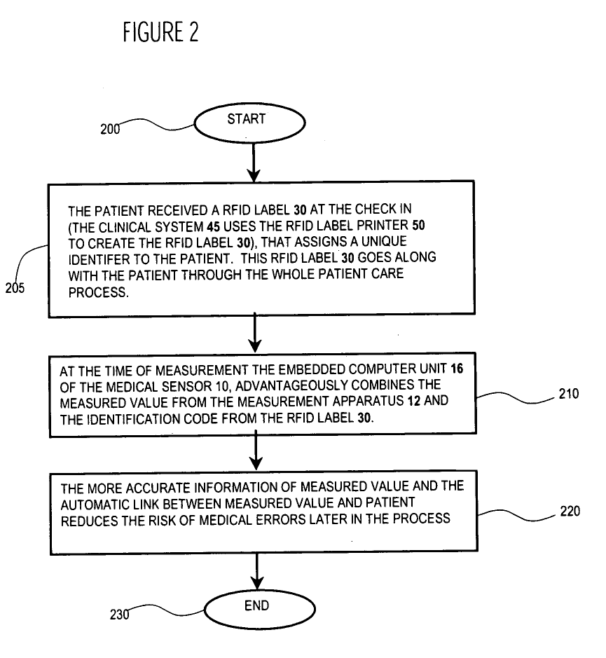

FIG. 2 shows a flowchart of steps for processing patient parameters employed by the system of FIG. 1. In step 205 following the start at step 200, an RFID tag 30 that provides a unique identifier to a patient, is created by printer 50 and attached to a patient during admission at a healthcare facility. RFID tag 30 accompanies the patient throughout the patient care process. In step 210 embedded computer 16 advantageously links a measured weight value of the patient received from A/D unit 14 with ancillary data 25 comprising patient identification information from RFID tag 30. In step 220, the automatically linked patient parameter (measured patient weight value) and patient identifier are used by clinical information systems such as a computerized physician order entry system for initiating administration of a treatment. Thereby manual data entry error is eliminated which advantageously reduces inaccuracy and error in prescription and administration of medications, for example. The system provides an accurate current patient weight value automatically acquired and associated with an accurate patient identifier. This data is automatically transferred to a computerized physician order entry system to check or complete order information. These steps are performed without manual intervention. The accurate current weight value is advantageously used by the order entry system to calculate a correct dose value for a patient. This reduces risk of medical errors later in a medication administration process since medication doses are based on an accurate current measured patient weight value. The automatic process is particularly valuable for determining an accurate dose value for a child patient where the danger of prescribing an overdose of medication is magnified. The process of FIG. 2 terminates at step 230.

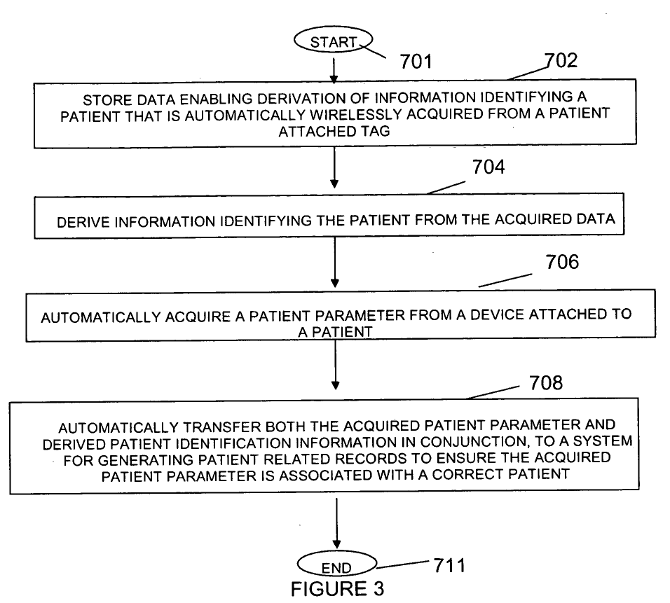

FIG. 3 shows a flowchart of steps involved in acquiring and processing patient parameters. In step 702 following the start at step 701, parameter acquisition device 10 (FIG. 1) stores data enabling derivation of information identifying a patient automatically wirelessly acquired from a patient attached tag 30. Patient attached tag 30 is an RFID compatible device and transceiver 20 automatically wirelessly acquires the data enabling derivation of information identifying the patient from the RFID tag. Device 10 derives information identifying the patient from the acquired data in step 704. Device 10 in step 706 automatically acquires measured patient values (e.g., weight, height, blood pressure, blood oxygen saturation, temperature, pulse rate, EKG, ECG or EEG data and respiration) from a device attached to a patient. In step 708, device 10 automatically transfers both the acquired patient parameter and derived patient identification information in conjunction, to a system (such as an order entry system) for generating patient related records and using the transferred parameter and identification information to ensure the acquired patient parameter is associated with a correct patient. Thereby, an order entry system automatically verifies an order is being generated for a correct patient and verifies an ordered medication dose is compatible with a measure patient weight value, for example. For this purpose, the order entry system compares a transferred acquired patient parameter value with an existing corresponding patient parameter value retrieved from a medical record associated with the patient identification information, to validate an order for a treatment related service is for the correct patient. In another embodiment, embedded computer unit 16 of device 10 may perform the comparison. The system may be employed by a variety of different medical parameter measurement devices including devices for determining blood pressure, pulse rate, respiration, weight: temperature, etc. The process of FIG. 3 terminates at step 711.

The system and processes presented in FIGS. 1-3 are not exclusive. Other systems and processes may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. Further, any of the functions provided by the system of FIG. 1 may be implemented in hardware, software or a combination of both.

1. A patient parameter processing system, comprising:
a transceiver for automatically wirelessly acquiring, from a patient attached tag, data enabling derivation of information identifying said patient;
an acquisition processor for automatically acquiring a patient parameter from a device attached to a patient; and
a data processor for automatically transferring both said acquired patient parameter and derived patient identification information in conjunction, to a system for generating patient related records and using said transferred parameter and identification information to ensure said acquired patient parameter is associated with a correct patient.

2. A system according to claim 1, including
an input processor for receiving said data enabling derivation of information identifying said patient and deriving said patient identification information.

3. A system according to claim 1, wherein
said data processor automatically transfers both said acquired patient parameter and derived patient identification information in conjunction, to an order entry system enabling automatic verification an order is being generated for a correct patient.

4. A system according to claim 3, wherein
at least one of, (a) said data processor and (b) said order entry system compares said transferred acquired patient parameter value with an existing corresponding patient parameter value to validate an order for a treatment related service is for the correct patient.

5. A system according to claim 3, wherein
said patient parameter is at least one of, (a) weight and (b) height.

6. A system according to claim 1, wherein
said patient parameter is at least one of, (a) blood pressure, (b) blood oxygen saturation, (c) temperature, (d) pulse rate, (e) EKG, ECG or EEG data and (f) a respiration parameter.

7. A system according to claim 1, wherein
said data processor automatically transfers both said acquired patient parameter and derived patient identification information in conjunction, to a patient medical record.

8. A system according to claim 1, wherein
said data processor compares said transferred acquired patient parameter value with an existing corresponding patient parameter value retrieved from a medical record associated with said patient identification information to validate correct patient identity.

9. A system according to claim 1, wherein
said patient attached tag is an RFID compatible device and said transceiver automatically wirelessly acquiring said data enabling derivation of information identifying said patient from said RFID tag.

10. A system according to claim 1, wherein
said patient parameter is a measured weight value and
said data processor automatically transfers both said acquired weight value and derived patient identification information in conjunction, to an order entry system enabling said order entry system to verify an ordered medication dose is compatible with said weight value.

11. A patient parameter processing system, comprising:
a transceiver for automatically wirelessly acquiring, from a patient attached tag, data enabling derivation of information identifying said patient;
an acquisition processor for automatically acquiring patient weight from a scale used to weigh said patient; and
a data processor for automatically transferring both said acquired weight value and derived patient identification information in conjunction, to a system for generating patient related records and using said transferred parameter and identification information to ensure said acquired patient parameter is associated with a correct patient.

12. A system according to claim 11, wherein
said scale is embodied within at least one of, (a) a patient bed, (b) a patient examination table, (c) a patient chair and (d) a couch used to support said patient.

13. A method for processing a patient parameter, comprising the activities of:
automatically wirelessly acquiring, from a patient attached tag, data enabling derivation of information identifying said patient;
automatically acquiring a patient parameter from a device attached to a patient; and
automatically transferring both said acquired patient parameter and derived patient identification information in conjunction, to a system for generating patient related records and using said transferred parameter and identification information to ensure said acquired patient parameter is associated with a correct patient.
